(19) United States
(12) Patent Application Publication
(10) Pub. No.: US 2023/0398006 A1
(43) Pub. Date: Dec. 14, 2023

(54) Neurovascular Stent

(71) Applicant: ACCUMEDICAL BEIJING LTD., Beijing (CN)

(72) Inventors: Tai Dac Tieu, Fountain Valley, CA (US); Jin Park, Morris Plains, NJ (US); Daniel Olsen, Califon, NJ (US); Cory Zegel, Hoboken, NJ (US)

(73) Assignee: ACCUMEDICAL BEIJING LTD., Beijing (CN)

(21) Appl. No.: 18/203,557

(22) Filed: May 30, 2023

Related U.S. Application Data
(60) Provisional application No. 63/351,103, filed on Jun. 10, 2022.
(60) Provisional application No. 63/394,376, filed on Aug. 2, 2022.

(51) Int. Cl. A61F 2/915

(52) U.S. Cl. CPC A61F 2/915; A61F 2002/91575

(57) ABSTRACT

A stent having a substantially tubular body is disclosed. In at least one embodiment, the stent provides a scaffold having a plurality of interconnected struts forming a plurality of radial axial rows of radial support cells and a plurality of flexible axial rows of flexible support cells. The plurality of radial axial rows and flexible axial rows are disposed in an alternating pattern along a circumferential axis of the stent. A center axis of each radial axial row and flexible axial row is parallel to a longitudinal axis of the stent. Each radial axial row is arranged so as to be offset along the longitudinal axis of the stent from each adjacent radial axial row. Each radial support cell is formed from four of the interconnected struts of the scaffold which are arranged so as to be symmetrical along both the longitudinal axis and the circumferential axis of the stent.

### RELATED APPLICATIONS

This application claims priority and is entitled to the filing date of U.S. provisional application Ser. No. 63/351,103, filed on Jun. 10, 2022. This application also claims priority to U.S. provisional application Ser. No. 63/394,376, filed on Aug. 2, 2022. The contents of the aforementioned applications are incorporated herein by reference.

### BACKGROUND

The subject of this patent application relates generally to the field of intravascular therapeutic devices, and more particularly to stents for treatment of vessel lumens that have been weakened by damage or disease, notably or treatment in the neurovasculature.

Applicant hereby incorporates herein by reference any and all patents and published patent applications cited or referred to in this application.

By way of background, blood vessel disorders, specifically those affecting the neurovasculature, including intracranial atherosclerotic disease (ICAD) and aneurysms are a significant point of interest for innovation. Per the Center for Disease Control and Prevention (CDC), in 2020, cerebrovascular diseases were the fifth highest cause of deaths of any cause in the United States.

One possible treatment of neurovascular disorders such as ICAD or aneurysms is placement of a stent at the diseased site. The implanted stent supports the weakened or damaged vessel which in turn helps to treat the disease in question. For example, a patient with ICAD will have an occluded or partially occluded vessel in the neurovasculature, which if present in a critical section of the anatomy will reduce or fully stop blood flow to the neurovasculature, resulting in stroke or death in severe cases. Treatment of ICAD may involve placement of a particularly small stent which is capable of supporting the diseased vessel, thereby allowing blood to flow unrestricted to distal portions of the vasculature, preventing future injury.

Current stent designs are made of a plurality of interconnected metallic struts, and come in one (1) of two (2) common forms, self-expanding, and balloon expandable. Self-expanding stents are made of materials which exhibit shape memory and super-elastic properties, such as Nitinol. The diameter of the stent aligns with the diameter of the blood vessel to be treated, and can range from 1.5 mm to 4.0 mm or more depending upon the desired anatomical location. Self-expanding stents are typically pre-loaded into a tube, such as an introducer sheath, which constrains the diameter of the stent until it has reached the final treatment location, at which time the diametrical constraint will be removed, allowing the stent to expand to its intended diameter, directly in contact with the vessel wall. Balloon expandable stents follow a similar methodology, however instead of being pre-loaded into a tube, the device is crimped onto a balloon catheter, and following delivery to the target location, is plastically deformed via expansion of the underlying balloon until proper stent apposition occurs.

Implantation of stents within the neurovasculature introduces several challenges, most notably the anatomical challenges of treating the small vessels of the neurovasculature, which are known to be tortuous. Therefore, for a stent to be appropriately designed to tackle the challenges presented by the neurovascular anatomy, the stent must be small and flexible, while still being able to provide the radial strength required to treat the diseased or damaged vessel. Additionally, as the stent is a permanent implant, the stent must be capable of withstanding repeated stresses typical to blood vessels and must be low profile in order to not increase risk of thrombus or other side effects of blood vessel implantation.

Stent flexibility is important as the stent is typically the stiffest part of the stent delivery system. The stent must be capable of not only traversing the tortuous paths of the anatomy, but also be capable of conforming to the treatment vessel geometry, which may include bends or twists. As such, a flexible stent is an absolute requirement to be able to treat a variety of blood vessel disorders.

Stent radial strength is critical as the role of the stent in the blood vessel is to provide structural support. The radial strength of the stent must be sufficient as not to be overcome by the radial compressive forces generated due to the pulsatile flow of blood, in addition to any additional forces exerted by the diseased area, such as plaque buildup. In the case of self-expanding stents, the stent radial strength must also be sufficient to open an occluded vessel to its intended diameter, requiring high levels of radial strength relative to the stent size.

The current trend in stent design is to provide a stent with as low of a profile as possible. It is understood that assuming stent flexibility and stent radial strength characteristics are constant, a stent with a lower profile will have better patient outcomes. In addition to the advantages that a low profile provides in terms of flexibility and vessel access, post implantation, a low-profile stent is less likely to induce thrombus or cause additional complications for the patient.

A variety of intravascular stents have been proposed in prior art, for example CN Patent Publication Number 106137481, U.S. Pat. Nos. 4,512,338, 4,733,665, 7,037,330, 7,695,507, and 8,518,102, and U.S. Patent Publication Number 2015/0209165; however, significant advances are still required to optimize radial strength in relation to stent profile.

For example, CN Patent Publication Number 106137481 discloses an intravascular stent containing multiple cell rings arranged in a tubular structure. The cell rings are composed of a first unit ring and a second unit ring, which are different in shape. Both the first unit ring and second unit ring are symmetrical around their center points. Both unit rings are at an angle θ relative to the horizontal axis, such that the cell rings form a helical structure when formed on a tube. The first unit ring has an angle β1 between the center line of the unit ring and the tangential axis of the stent strut. The second unit ring has an angle β2 formed in the same manner. The prior art theorizes that β1>β2. The prior art theorizes that this intravascular stent is formed from a laser cut shape memory alloy such as nitinol. CN Patent Publication Number 106137481 discloses that the design will have good radial force and flexibility characteristics, making it practical for clinical use. However, this design requires the stent to deploy in a segmented manner, with each column of cells deploying individually, in a non-uniform and more traumatic manner. Further, the angle θ causes the stent to have a helical structure so that the rows of unit rings will be spiral along the length of the stent. As the stent bends it will cause a twisting motion, which causes additional relative motion against the vessel wall. Moreover, the stent design disclosed in CN Patent Publication Number 106137481 does not differentiate between loading conditions for radial and longitudinal loading, which limits the amount of radial force or strength that can be applied to the stent.

Conventional solutions to improving radial strength require increased stent geometry (increased wall thickness or strut width), which reduces stent flexibility and increases crossing profile. It stands to reason that a stent design which provided greater radial strength than current stent designs while maintaining stent flexibility and a low crossing profile would be advantageous, as, in order to align with current anticipated radial strength characteristics, the stent geometry (e.g., wall thickness, strut width) can be reduced while still providing the expected level of radial strength and flexibility.

Therefore, there is a current need for a stent which will provide a high radial strength without compromising on flexibility and which allows for a lower-profile design. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a stent having a substantially tubular body is disclosed and configured for having a relatively lower-profile design, providing a relatively higher radial strength without compromising on flexibility. In at least one embodiment, the stent provides a scaffold having a plurality of interconnected struts forming a plurality of radial axial rows of radial support cells and a plurality of flexible axial rows of flexible support cells. The plurality of radial axial rows and the plurality of flexible axial rows are disposed in an alternating pattern along a circumferential axis of the stent. A center axis of each radial axial row and flexible axial row is parallel to a longitudinal axis of the stent. Each radial axial row is arranged so as to be offset along the longitudinal axis of the stent from each adjacent radial axial row. Each radial support cell is formed from four of the plurality of interconnected struts of the scaffold which are arranged so as to be symmetrical along both the longitudinal axis and the circumferential axis of the stent.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a flattened view of a plurality of interconnected struts forming an exemplary stent, in an unconstrained state, in accordance with at least one embodiment;

FIG. 2A is a flattened view of the stent of FIG. 1, in a radially constrained state, in accordance with at least one embodiment;

FIG. 2B is a flattened view of the stent of FIG. 1, in a longitudinally constrained state, in accordance with at least one embodiment;

FIG. 3 is a tubular view of the stent of FIG. 1, in accordance with at least one embodiment;

FIG. 4 is a view of an exemplary radial support cell of the stent in a radially constrained state with a vertical strut angle of −29°, in accordance with at least one embodiment;

FIG. 5 is a view of an exemplary radial support cell of the stent in an unconstrained state with a vertical strut angle of 0°, in accordance with at least one embodiment;

FIG. 6 is a view of an exemplary radial support cell of the stent in a longitudinally constrained state with a vertical strut angle of 45°, in accordance with at least one embodiment;

FIG. 7 is a view of an exemplary radial support cell of the stent in a longitudinally constrained state with a vertical strut angle of 58°, in accordance with at least one embodiment;

FIG. 8 is a view of an exemplary radial support cell of the stent in a longitudinally constrained state with a vertical strut angle of 76°, in accordance with at least one embodiment;

FIG. 9 is a view of an exemplary radial support cell of the stent in a longitudinally constrained state with a vertical strut angle of 83°, in accordance with at least one embodiment;

FIG. 10 is a view of an exemplary radial support cell a longitudinally constrained state with a vertical strut angle of 89°, in accordance with at least one embodiment;

FIG. 11 is a view of an exemplary radial support cell of the stent in a longitudinally constrained state with a vertical strut angle of 99°, in accordance with at least one embodiment;

FIG. 12 is a graph of the approximate radial force (“RF”) in pounds exhibited by an exemplary radial support cell of the stent with a given vertical strut angle for varying levels of displacement, in accordance with at least one embodiment;

FIG. 13 is a graph of the approximate vertical stent cell angle plotted against stent stiffness, in accordance with at least one embodiment;

FIG. 14 is a flattened view of a plurality of interconnected struts forming a further exemplary stent, in an unconstrained state, in accordance with at least one embodiment;

FIG. 14A is an exemplary detailed view of the circled section defined in FIG. 14, in accordance with at least one embodiment;

FIG. 14B is an alternate exemplary detailed view of the circled section defined in FIG. 14, in accordance with at least one embodiment; and

FIG. 14C is a further alternate exemplary detailed view of the circled section defined in FIG. 14, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

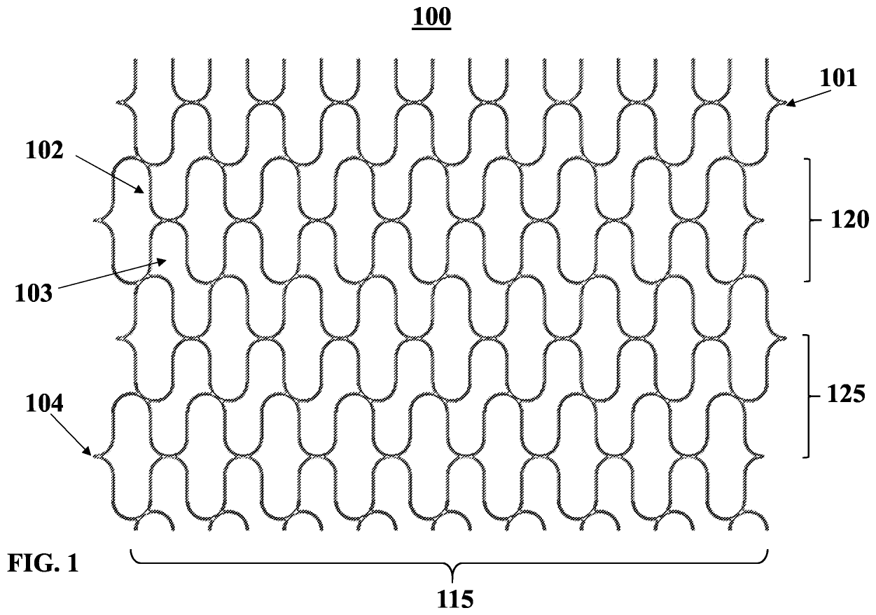

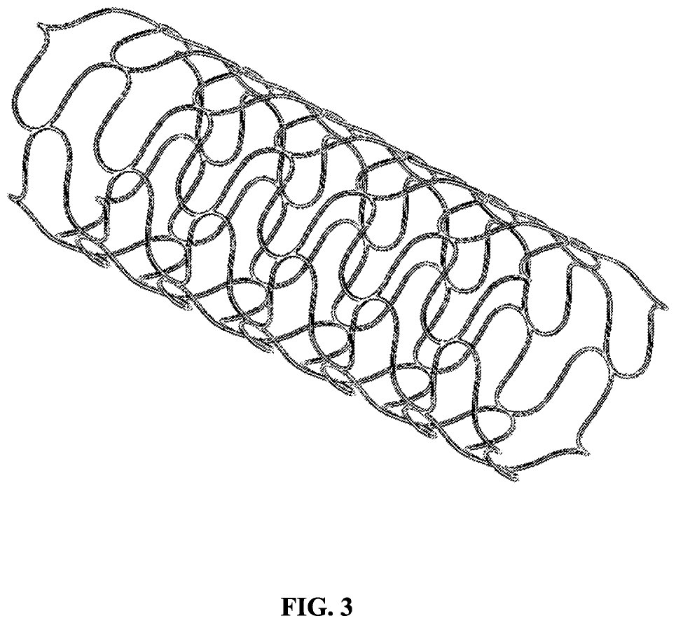

Turning now to FIG. 1, there is shown a flattened view of a plurality of interconnected struts forming a stent 100, in an unconstrained state, according to at least one embodiment. The view in FIG. 1 is a flat representation of the stent 100, which, in its final form, would be wrapped into a cylindrical/tubular design, similar to that shown in FIG. 3. The unconstrained state of the stent 100 corresponds to no radial load and no longitudinal load being applied to the stent 100. Thus, the unconstrained state represents a condition where the stent 100 has no forces acting upon it, and is in an equilibrium or “memory” state. At the outset, it should be noted that the embodiments of the stent 100 depicted in the drawings are merely exemplary and are shown for illustrative purposes. Accordingly, in further embodiments, the stent 100 (along with each of the components of the stent 100 described herein) may take on any other sizes, shapes, dimensions and/or configurations now known or later developed—dependent at least in part on the specific context in which the stent 100 is to be utilized—so long as the stent 100 is able to substantially carry out the functionality described herein.

In at least one embodiment, the stent 100 is made from a single piece of laser cut material that exhibits shape memory and super-elastic properties, such as nitinol for example; however, in further embodiments, the stent 100 may be constructed out of any other material (or combination of materials) now known or later developed, that allows the stent 100 to substantially carry out the functionality described herein.

In at least one embodiment, the stent 100 includes a plurality of sections, a proximal end 104, a distal end 101, and a scaffold comprising a plurality of radial support cells 102 and a plurality of flexible support cells 103. In at least one embodiment, the proximal end 104 of the stent 100 has an open design instead of a closed design in order to provide a potential location of attachment to a corresponding delivery system.

In at least one embodiment, the stent 100 provides an at least one radiopaque element 116 (e.g., marker) positioned and configured for improving visibility and ease of use during an insertion or deployment procedure. In at least one embodiment, the at least one radiopaque element 116 is positioned on one or both of the proximal end 104 and distal end 101 of the stent 100. In at least one embodiment, the at least one radiopaque element 116 is positioned elsewhere along the length (scaffold 115) of the stent 100. In at least one such embodiment, the at least one radiopaque element 116 is constructed out of a tungsten-loaded polymer. In further embodiments, the at least one radiopaque element 116 may be constructed out of platinum, chromium, cobalt, tantalum, nitinol, gold, silver, bismuth subcarbonate, barium sulfate, bismuth oxychloride, bismuth trioxide, stainless steel or alloys thereof, or any other radiopaque material (or combination of materials) now known or later developed.

In at least one embodiment, the distal end 101 of the stent 100 is similar in shape to a radial support cell 102, except that the distal ends 101 terminate to rounded edges as opposed to attaching to continuous aspects of the stent 100 design. In at least one embodiment, both proximal and distal ends 104 and 101 of the stent 100 may terminate at the end of a repeating junction, such that the entire stent 100 body is symmetrical. In at least one alternate embodiment, one or both of the proximal and distal ends 104 and 101 of the stent 100 may terminate with open designs to provide placement for additional radiopaque elements 116, attachments to delivery systems, or to provide additional atraumatic edges.

In at least one embodiment, each radial support cell 102 is an arrangement of four struts, where the struts are symmetrical along both an x-axis (longitudinal axis) and a y-axis (circumferential axis) of the stent 100. In at least one embodiment, the radial support cells 102 repeat in radial axial rows 120 along the length of the stent 100. Additionally, in at least one embodiment, each flexible support cell 103 is an arrangement of four struts, with the flexible support cells 103 repeating in flexible axial rows 125, adjacent to the radial axial rows 120 of radial support cells 102, along the length of the stent 100. In at least one embodiment, each flexible support cell 103 has mirror symmetry to a corresponding flexible support cell 103 in another flexible axial row 125, in the circumferential direction, of flexible support cells 103. In at least one embodiment, the radial axial rows 120 of the radial support cells 102 alternate with the flexible axial rows 125 of flexible support cells 103 circumferentially around the stent 100.

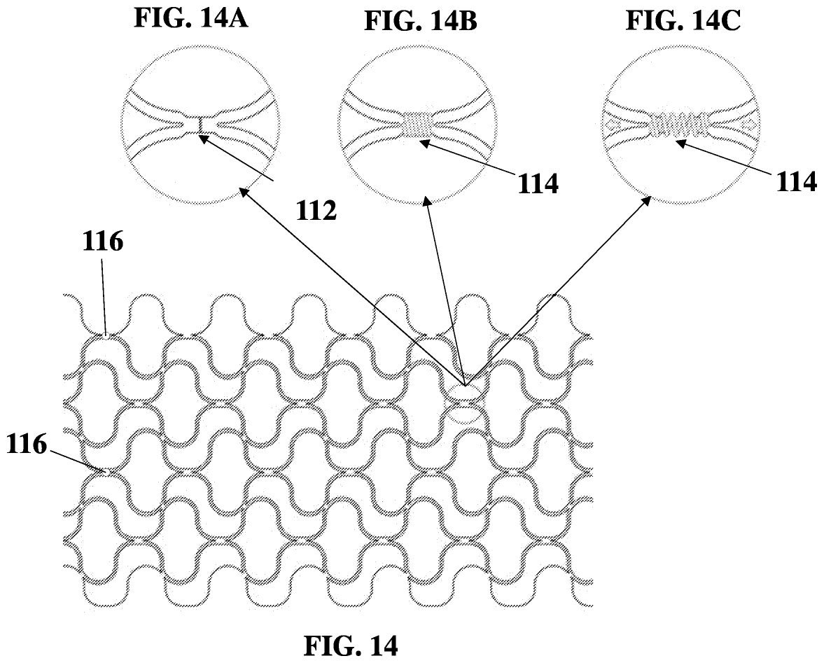

In at least one embodiment, the radial support cells 102 and flexible support cells 103 are formed by uninterrupted material so as to form a stent 100 of unitary construction. In at least one alternate embodiment, as illustrated in FIGS. 14 and 14A, adjacent ones of the radial support cells 102 and flexible support cells 103 are interrupted by an intentional break 112 therebetween so as to create a non-continuous geometry. In at least one such embodiment, the break 112 is then reconnected through the use of a coil 114. Such a connection via the coil 114 allows for multi-axial movement of the joint to maximize stent 100 flexibility when positioned within a target treatment vessel. In at least one embodiment, each coil 114 is constructed out of a tungsten-loaded polymer. In further embodiments, the each coil 114 may be constructed out of platinum, platinum iridium, chromium, cobalt, tantalum, nitinol, a nitinol composite, gold, silver, bismuth subcarbonate, barium sulfate, bismuth oxychloride, bismuth trioxide, stainless steel or alloys thereof, or any other radiopaque material (or combination of materials) now known or later developed.

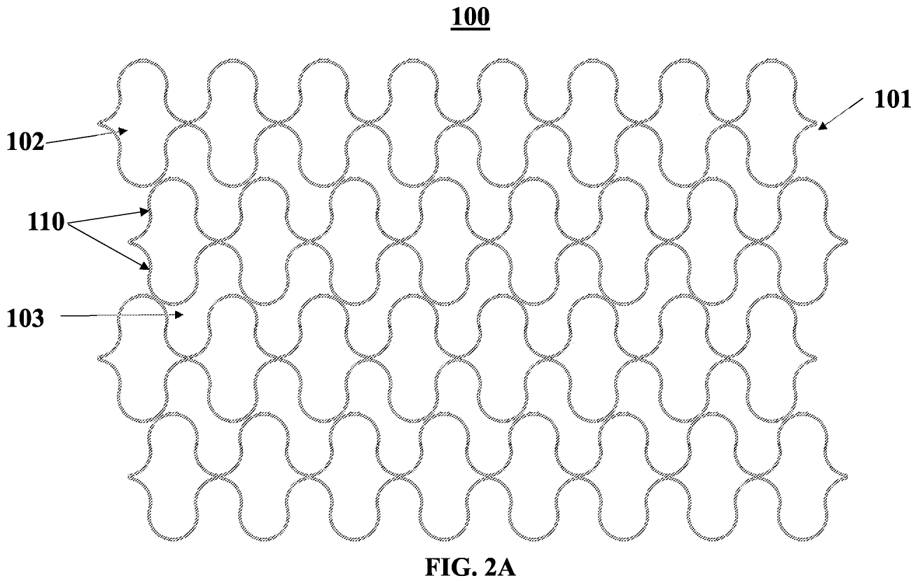

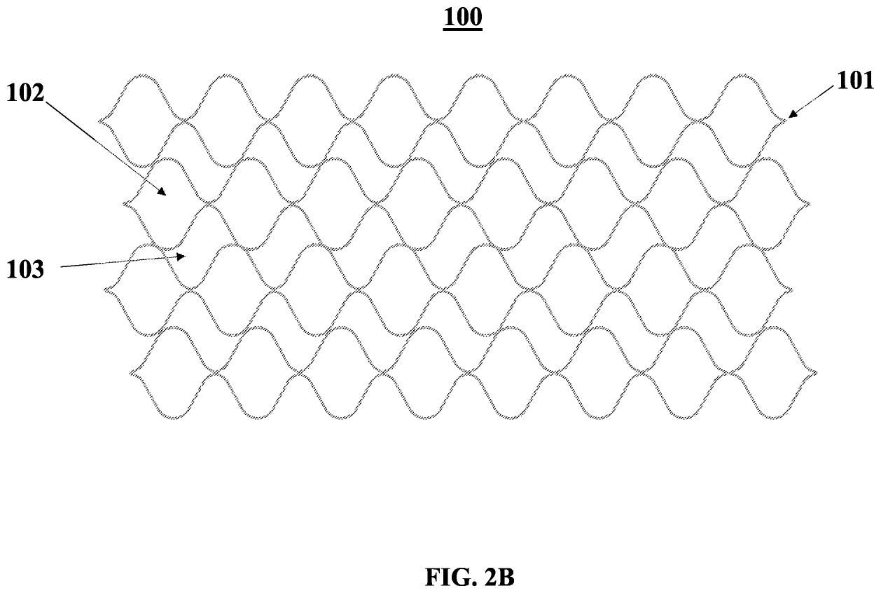

FIG. 2A shows a flattened view of the stent 100 of FIG. 1 in a radially constrained state. In at least one embodiment, when a radial load or radial force acts upon the stent 100, opposing vertical struts 110 of each radial support cell 102 buckle inwardly toward one another. In contrast to the radially constrained state, in the unconstrained state shown in FIG. 1, opposing vertical struts 110 of each radial support cell 102 are substantially perpendicular to a central axis of the radial axial row 120 of radial support cells 102. Further, as the central axes of the radial axial rows 120 are parallel to the longitudinal axis of the stent 100, the vertical struts 110 are also substantially perpendicular to the longitudinal axis of the stent 100 in the unconstrained state. In at least one alternate embodiment, the vertical struts 110 of each radial support cell 102 are less than perpendicular with a vertical strut angle of less than 0° in an unconstrained state to promote buckling while under a radial load or radial force.

FIG. 2B shows a flattened view of the stent 100 of FIG. 1 in a longitudinally constrained state. In at least one embodiment, when an axial tension or longitudinal force acts upon the stent 100, the vertical struts 110 of each radial support cell 102 deform outwardly from one another.

In at least one embodiment, the flexible support cells 103 exist as an axial (horizontal) row of cells to separate the radial support cells 102. Additionally, in at least one embodiment, the shape of the flexible support cells 103 permits the adjacent radial support cells 102 to be spaced apart and offset from one another in the circumferential direction, which further improves the flexibility of the stent 100 while maintaining radial strength.

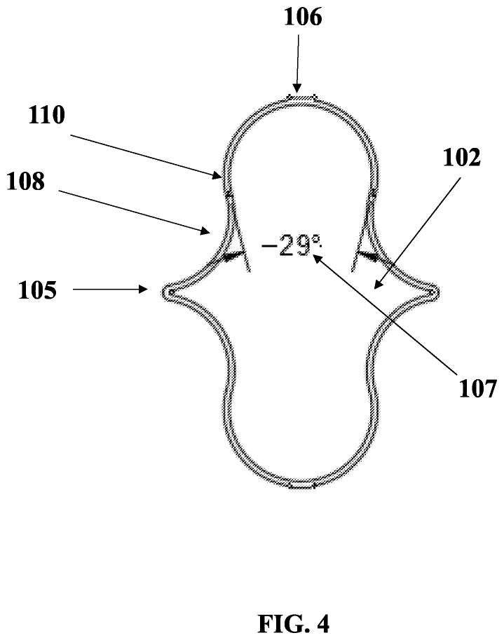

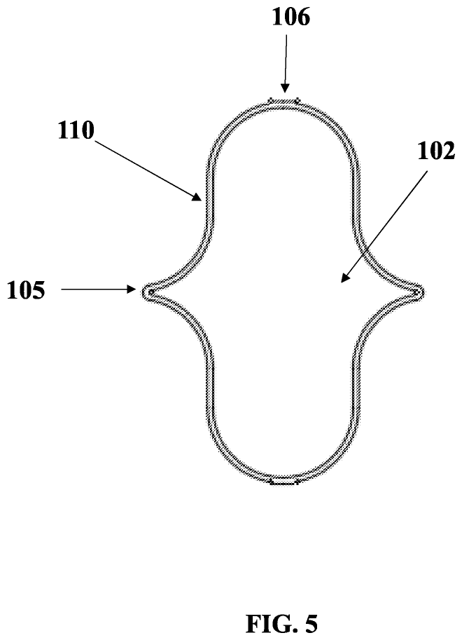

FIG. 4 depicts a flattened view of a radial support cell 102 in a radially constrained state. In at least one embodiment, each radial support cell 102 includes four vertical struts 110 and defines a joint apex 105, a top radius 106, and a vertical strut angle 107. In at least one embodiment, each vertical strut 110 is in a substantially vertical or less than vertical configuration while in an unconstrained or unloaded state, as shown in FIG. 5. While under the unconstrained configuration shown in FIG. 5, each radial support cell 102 is capable of adapting to different types of applied loads, such as a radial compression or load that is observed in a blood vessel, or an axial tensile stress that is observed while loading the stent 100 into a delivery system. In the unconstrained configuration, the geometry of each radial support cell 102 (including the joint apex 105 and the top radius 106) has gentle radius edges to further support flexibility and conformability to the tubular nature of blood vessels.

In at least one embodiment, when the stent 100 is implanted into a blood vessel, the blood vessel will radially compress the stent 100. The continued radial force exerted by the stent 100 on the blood vessel prevents the blood vessel from collapsing or occluding, ensuring that blood may flow freely down the treated vessel. The continued radial force exerted by the stent 100 on the blood vessel also ensures that the stent 100 will properly adhere to the vessel wall to prevent movement both during the implantation procedure and post implantation. In at least one embodiment, as the stent 100 is radially compressed by the blood vessel, the vertical struts 110 of each radial support cell 102 will begin to buckle inwards, creating a vertical strut angle 107 of less than 0°, as shown in FIG. 4. This buckling action increases the radial strength of the stent 100. As the radial strength is derived from the buckling action, the radial strength is high in relation to the geometry of the individual vertical struts 110. As such, to align the stent 100 with commonly accepted radial strength values, the stent geometry (e.g., wall thickness, strut width) can be proportionately reduced. By reducing the stent geometry dimensions, the overall crossing profile may be reduced. Additionally, the footprint of the stent 100 on the treatment vessel will be reduced, which may accelerate healing in vivo.

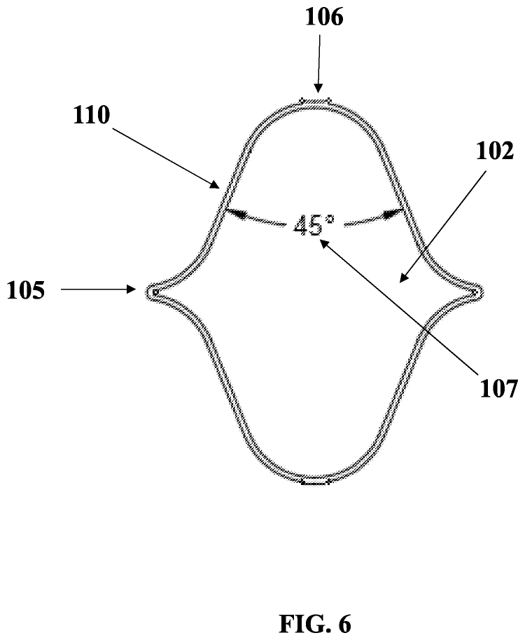

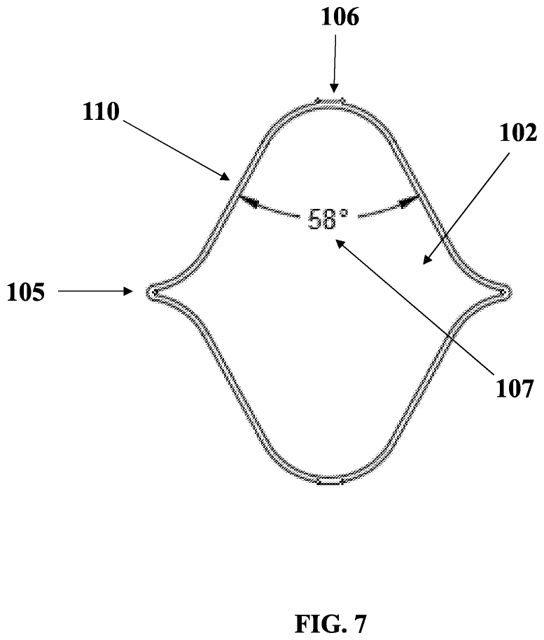

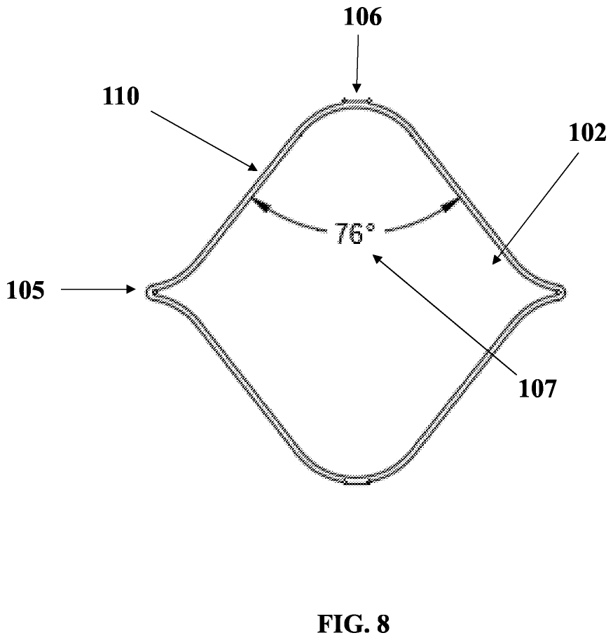

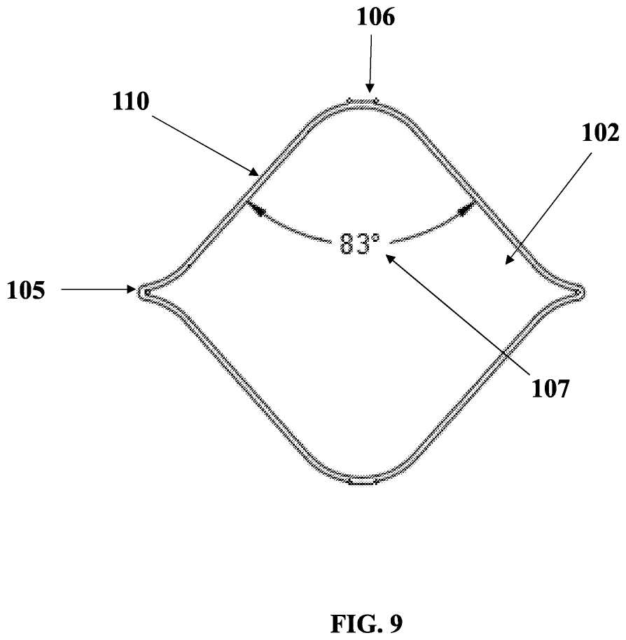

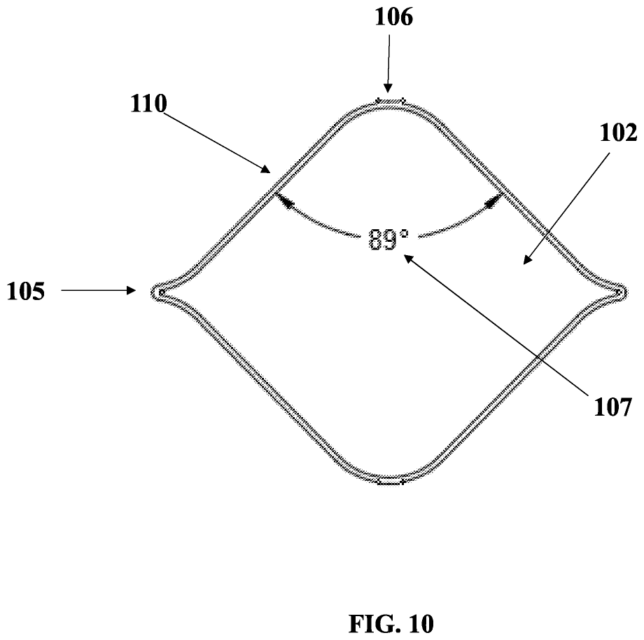

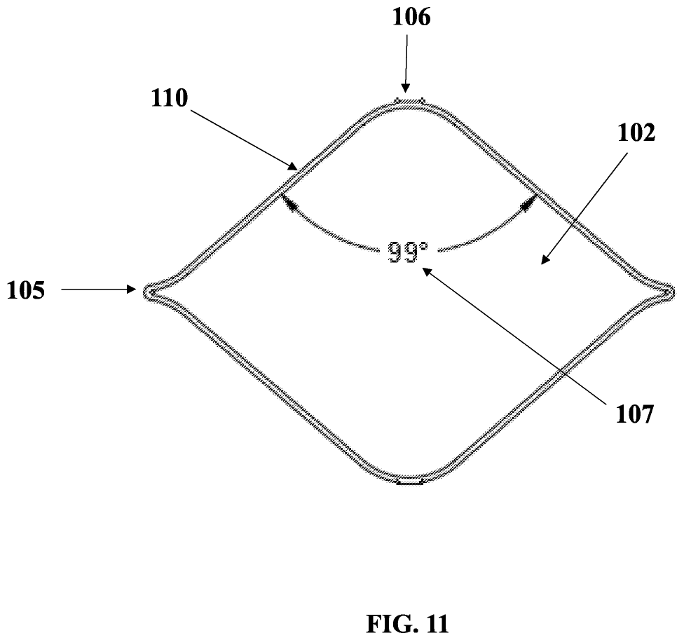

In at least one embodiment, when a given radial support cell 102 is in the radially loaded configuration shown in FIG. 4, all aspects of the geometry remain rounded, with the top radius 106 and a vertical strut radius 108 maximized to reduce internal stress and strain within the design, and therefore minimize the risk of high stress failure. Conversely, the strong radial strength provided by the radial support cells 102 may be countered by applying a tensile load along an axis of two opposing joint apices 105. This allows the stent 100 to be loaded into a delivery system without the need to combat the high radial strength observed during the buckling in FIG. 4. FIGS. 6-11 show a radial support cell 102 with varying vertical strut angles 107 ranging from about 45° to about 99°, as increasing amounts of axial tension or longitudinal force is applied to the radial support cell 102. As a tensile load is applied to the stent 100, the vertical strut angle 107 will increase, and in turn, the radial strength provided by the radial support cell 102 will decrease. This allows a simple and relatively low stress method of loading the stent 100 into a sheath or delivery system. In the tensile loaded configuration depicted in FIGS. 6-11, all aspects of the geometry remain rounded, with the bulk of the vertical strut 110 movement captured within the top apex 106.

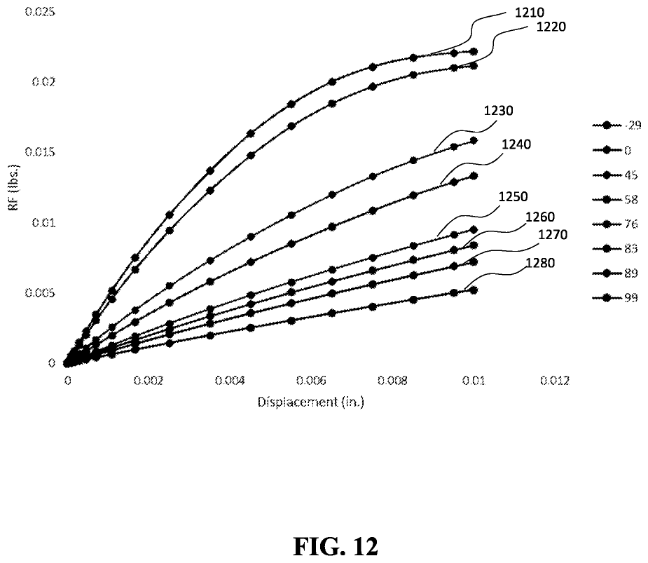

FIG. 12 shows a graphical representation of the radial force (“RF”) in pounds provided by each radial support cell 102 for a given displacement in at least one embodiment. Eight different lines are presented in the graph, each representing a radial support cell 102 with a different vertical strut angle 107. The top line 1210 corresponds to a vertical strut angle of −29°, while the stent 100 is in the radially constrained state. Line 1220 corresponds to a vertical strut angle of 0°, while the stent 100 is in the unconstrained state. Line 1230 corresponds to a vertical strut angle of 45°, while the stent 100 is in the longitudinally constrained state. Line 1240 corresponds to a vertical strut angle of 58°, while the stent 100 is in the longitudinally constrained state. Line 1250 corresponds to a vertical strut angle of 76°, while the stent 100 is in the longitudinally constrained state. Line 1260 corresponds to a vertical strut angle of 83°, while the stent 100 is in the longitudinally constrained state. Line 1270 corresponds to a vertical strut angle of 89°, while the stent 100 is in the longitudinally constrained state. Line 1280 corresponds to a vertical strut angle of 99°, while the stent 100 is in the longitudinally constrained state. In some embodiments of the invention, the vertical strut angle 107 may be less than 0° in the unconstrained state. The graph shows that, at high displacements of approximately 0.01 inches, the corresponding radial forces for a vertical strut angle 107 of 0° or −29° are more than double the radial forces corresponding to vertical strut angles 107 of 76° or more. Overall, as the vertical strut angle 107 increases, the radial force decreases drastically. This allows the stent 100 to have increased flexibility while in the longitudinally constrained state, during deployment in a blood vessel (lumen).

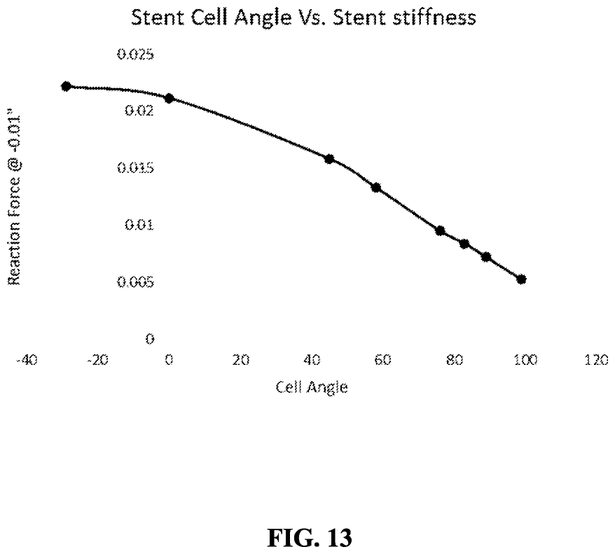

FIG. 13 shows a graphical representation of a stiffness of the stent 100 (i.e., flexibility) compared against the vertical strut angle 107 in at least one embodiment. As shown in FIG. 13, the stent stiffness, as measured by a stent reaction force, decreases as the vertical strut angle 107 increases with increasing longitudinal load or axial tension. This observation further demonstrates that the radial support cell 102 is capable of different properties (improved strength and improved flexibility) depending upon its configuration (radially constrained or longitudinally constrained).

The above-discussed features of exemplary embodiments of the stent 100 provide significant, non-obvious improvements and advantages over known prior art stents. For example, while the stent described in CN Patent Publication Number 106137481 comprises radial support cells and flexible support cells that would allow the stent to be functional for large size lumens, the design is based on an angle of the first unit ring being larger than an angle of the second unit ring. In contrast, in at least one embodiment of the stent 100, the cell angles corresponding to the angles disclosed in CN Patent Publication Number 106137481 are unimportant.

Instead, at least one embodiment of the stent 100 is based on the discovery that the vertical strut angle 107 plays a critical role in dramatically increasing both the radial strength characteristics and the flexibility of the stent 100. In at least one embodiment, the vertical strut angle 107 is designed to be variable based upon the type of load applied to the stent 100, giving different loading conditions for radial and longitudinal loading. When under radial compression, the vertical struts 110 will buckle or invert, drastically increasing radial force. While under longitudinal loading, the vertical struts 110 will extend, drastically decreasing radial force. In comparison, the known prior art provides no distinction between radial and longitudinal loading.

Second, the stent described in CN Patent Publication Number 106137481 comprises rows of unit rings which are stacked circumferentially upon one another in a radial configuration or vertically upon one another in a two-dimensional configuration. Thus, the circumferential axis of each unit ring is aligned along one axis. The aligned arrange causes the stent to deploy in a segmented manner, with each column of cells deploying individually.

In contrast, in at least one embodiment, each radial support cell 102 (corresponding to a unit ring) is staggered or offset along the circumferential axis such that the adjacent radial axial rows 120 of radial support cells 102 do not share an aligned center axis. This staggered arrangement ensures that the stent 100 will deploy in a continuous manner, with each column of radial support cells 102 deploying along with adjacent radial support cells 102. This difference in stent designs permits a more uniform and atraumatic deployment when compared to CN Patent Publication Number 106137481.

Third, although CN Patent Publication Number 106137481 discloses a row of unit rings, where the unit rings are aligned in a linear design along a line at angle θ, the stent has a helical structure so that the rows of unit rings spiral along the length of the stent. In contrast, in at least one embodiment, each radial support cell 102 (corresponding to a unit ring) within a radial axial row 120 is aligned in a linear design along a line that is parallel to the central axis of the stent 100, and therefore does not have any angle or helical structure. In CN Patent Publication Number 106137481, as the stent bends to move through the blood vessel, it will cause a twisting motion which may cause additional relative motion against the vessel wall. In at least one embodiment, the design of the stent 100 ensures that the stent 100 will simply bend without any twisting motion and thereby minimize any relative motion against the vessel wall.

These differences in stent design cause very different behavior. In CN Patent Publication Number 106137481, the stent will react similarly under both longitudinal and radial load, such that the rows of unit rings, or cells, will compress and lengthen relative to the central axis of the stent. At least one embodiment of the stent 100, on the other hand, reacts differently to longitudinal and radial loads, having a response optimized for each condition. Longitudinal loads are typically applied during loading or retraction, where the desired radial force is low. Radial loads are typically applied after the stent 100 has reached its intended treatment location and is in contact with the vessel wall, where the desired radial force is high. In at least one embodiment, under radial loading, the vertical struts 110 will invert or buckle, resulting in a negative vertical strut angle 107, thereby increasing the stent radial force dramatically. In this manner, in at least one embodiment, the stent 100 provides a mechanism to give high radial force under radial load and low radial force under longitudinal loads, while known prior art stents do not provide a distinction between the two types of loads.

Aspects of the present specification may also be described as the following embodiments:

1. A stent having a substantially tubular body comprising: a proximal end and an opposing distal end; and a scaffold having a plurality of interconnected struts forming a plurality of radial axial rows of radial support cells and a plurality of flexible axial rows of flexible support cells; wherein the plurality of radial axial rows and the plurality of flexible axial rows are disposed in an alternating pattern along a circumferential axis of the stent; wherein a center axis of each radial axial row and flexible axial row is parallel to a longitudinal axis of the stent; wherein each radial axial row is arranged so as to be offset along the longitudinal axis of the stent from each adjacent radial axial row; and wherein each radial support cell is formed from four of the plurality of interconnected struts of the scaffold which are arranged so as to be symmetrical along both the longitudinal axis and the circumferential axis of the stent.

2. The stent according to embodiment 1, wherein each flexible support cell is formed from four of the plurality of interconnected struts of the scaffold which are arranged such that two adjacent flexible axial rows have mirror symmetry along the longitudinal axis of the stent.

3. The stent according to embodiments 1-2, wherein, for each of the radial support cells, the four struts that form said radial support cells are disposed such that a pair of opposing vertical struts are oriented substantially perpendicular to the longitudinal axis of the stent.

4. The stent according to embodiments 1-3, wherein the vertical struts of each radial support cell is configured for buckling inwardly toward one another when exposed to a radial force.

5. The stent according to embodiments 1-4, wherein the vertical struts of each radial support cell are configured for deforming from a linear shape to a curved shape having a large radius when said vertical struts are exposed to the radial force.

6. The stent according to embodiments 1-5, wherein the vertical struts of each radial support cell is configured for deform outwardly from one another when exposed to a tensile force while the stent is in a longitudinally constrained configuration.

7. The stent according to embodiments 1-6, wherein an angle between the struts is greater than 0° when the stent is in the longitudinally constrained configuration.

8. The stent according to embodiments 1-7, wherein a radial force exerted by the stent is lower when the stent is in the longitudinally constrained configuration than when the stent is in each of an unconstrained configuration and a radially constrained configuration.

9. The stent according to embodiments 1-8, wherein the radial force exerted by the stent when the stent is in the longitudinally constrained configuration permits the stent to be compressed and loaded into a stent delivery system for insertion into a body lumen.

10. The stent according to embodiments 1-9, further comprising an at least one radiopaque element formed on the stent.

11. The stent according to embodiments 1-10, wherein the at least one radiopaque element is positioned on one or both of the proximal end and distal end of the stent.

12. The stent according to embodiments 1-11, wherein the at least one radiopaque element is positioned on a length of the stent.

13. The stent according to embodiments 1-12, wherein the at least one radiopaque element is constructed out of at least one of a tungsten-loaded polymer, platinum, chromium, cobalt, tantalum, nitinol, gold, silver, bismuth subcarbonate, barium sulfate, bismuth oxychloride, bismuth trioxide, stainless steel or alloys thereof.

14. The stent according to embodiments 1-13, wherein: adjacent ones of the radial support cells are interrupted by a break therebetween; and adjacent ones of the radial support cells are re-connected by a coil spanning the break therebetween.

15. The stent according to embodiments 1-14, wherein each coil is constructed out of at least one of a tungsten-loaded polymer, platinum, platinum iridium, chromium, cobalt, tantalum, nitinol, a nitinol composite, gold, silver, bismuth subcarbonate, barium sulfate, bismuth oxychloride, bismuth trioxide, stainless steel or alloys thereof.

16. The stent according to embodiments 1-15, wherein the stent is constructed out of a single piece of laser cut material that exhibits shape memory and super-elastic properties.

17. A stent having a substantially tubular body comprising: a proximal end and an opposing distal end; and a scaffold having a plurality of interconnected struts forming a plurality of radial axial rows of radial support cells and a plurality of flexible axial rows of flexible support cells; wherein the plurality of radial axial rows and the plurality of flexible axial rows are disposed in an alternating pattern along a circumferential axis of the stent; wherein a center axis of each radial axial row and flexible axial row is parallel to a longitudinal axis of the stent; wherein each radial axial row is arranged so as to be offset along the longitudinal axis of the stent from each adjacent radial axial row; wherein each radial support cell is formed from four of the plurality of interconnected struts of the scaffold which are arranged so as to be symmetrical along both the longitudinal axis and the circumferential axis of the stent; and wherein each flexible support cell is formed from four of the plurality of interconnected struts of the scaffold which are arranged such that two adjacent flexible axial rows have mirror symmetry along the longitudinal axis of the stent.

18. A stent having a substantially tubular body comprising: a proximal end and an opposing distal end; a scaffold having a plurality of interconnected struts forming a plurality of radial axial rows of radial support cells and a plurality of flexible axial rows of flexible support cells; and an at least one radiopaque element formed on the stent; wherein the plurality of radial axial rows and the plurality of flexible axial rows are disposed in an alternating pattern along a circumferential axis of the stent; wherein a center axis of each radial axial row and flexible axial row is parallel to a longitudinal axis of the stent; wherein each radial axial row is arranged so as to be offset along the longitudinal axis of the stent from each adjacent radial axial row; and wherein each radial support cell is formed from four of the plurality of interconnected struts of the scaffold which are arranged so as to be symmetrical along both the longitudinal axis and the circumferential axis of the stent.

In closing, regarding the exemplary embodiments of the present invention as shown and described herein, it will be appreciated that a neurovascular stent is disclosed and configured for having a relatively lower-profile design, providing a relatively higher radial strength without compromising on flexibility. Because the principles of the invention may be practiced in a number of configurations beyond those shown and described, it is to be understood that the invention is not in any way limited by the exemplary embodiments, but is generally directed to a neurovascular stent and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that any methods disclosed herein, along with the order in which the respective elements of any such method are performed, are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A stent having a substantially tubular body comprising:
a proximal end and an opposing distal end; and
a scaffold having a plurality of interconnected struts forming a plurality of radial axial rows of radial support cells and a plurality of flexible axial rows of flexible support cells;
wherein the plurality of radial axial rows and the plurality of flexible axial rows are disposed in an alternating pattern along a circumferential axis of the stent;
wherein a center axis of each radial axial row and flexible axial row is parallel to a longitudinal axis of the stent;
wherein each radial axial row is arranged so as to be offset along the longitudinal axis of the stent from each adjacent radial axial row; and
wherein each radial support cell is formed from four of the plurality of interconnected struts of the scaffold which are arranged so as to be symmetrical along both the longitudinal axis and the circumferential axis of the stent.

2. The stent of claim 1, wherein each flexible support cell is formed from four of the plurality of interconnected struts of the scaffold which are arranged such that two adjacent flexible axial rows have mirror symmetry along the longitudinal axis of the stent.

3. The stent of claim 1, wherein, for each of the radial support cells, the four struts that form said radial support cells are disposed such that a pair of opposing vertical struts are oriented substantially perpendicular to the longitudinal axis of the stent.

4. The stent of claim 3, wherein the vertical struts of each radial support cell is configured for buckling inwardly toward one another when exposed to a radial force.

5. The stent of claim 4, wherein the vertical struts of each radial support cell are configured for deforming from a linear shape to a curved shape having a large radius when said vertical struts are exposed to the radial force.

6. The stent of claim 3, wherein the vertical struts of each radial support cell is configured for deform outwardly from one another when exposed to a tensile force while the stent is in a longitudinally constrained configuration.

7. The stent of claim 6, wherein an angle between the struts is greater than 0° when the stent is in the longitudinally constrained configuration.

8. The stent of claim 6, wherein a radial force exerted by the stent is lower when the stent is in the longitudinally constrained configuration than when the stent is in each of an unconstrained configuration and a radially constrained configuration.

9. The stent of claim 8, wherein the radial force exerted by the stent when the stent is in the longitudinally constrained configuration permits the stent to be compressed and loaded into a stent delivery system for insertion into a body lumen.

10. The stent of claim 1, further comprising an at least one radiopaque element formed on the stent.

11. The stent of claim 10, wherein the at least one radiopaque element is positioned on one or both of the proximal end and distal end of the stent.

12. The stent of claim 10, wherein the at least one radiopaque element is positioned on a length of the stent.

13. The stent of claim 10, wherein the at least one radiopaque element is constructed out of at least one of a tungsten-loaded polymer, platinum, chromium, cobalt, tantalum, nitinol, gold, silver, bismuth subcarbonate, barium sulfate, bismuth oxychloride, bismuth trioxide, stainless steel or alloys thereof.

14. The stent of claim 1, wherein:
adjacent ones of the radial support cells are interrupted by a break therebetween; and
adjacent ones of the radial support cells are re-connected by a coil spanning the break therebetween.

15. The stent of claim 14, wherein each coil is constructed out of at least one of a tungsten-loaded polymer, platinum, platinum iridium, chromium, cobalt, tantalum, nitinol, a nitinol composite, gold, silver, bismuth subcarbonate, barium sulfate, bismuth oxychloride, bismuth trioxide, stainless steel or alloys thereof.

16. The stent of claim 1, wherein the stent is constructed out of a single piece of laser cut material that exhibits shape memory and super-elastic properties.

17. A stent having a substantially tubular body comprising:
a proximal end and an opposing distal end; and
a scaffold having a plurality of interconnected struts forming a plurality of radial axial rows of radial support cells and a plurality of flexible axial rows of flexible support cells;
wherein the plurality of radial axial rows and the plurality of flexible axial rows are disposed in an alternating pattern along a circumferential axis of the stent;
wherein a center axis of each radial axial row and flexible axial row is parallel to a longitudinal axis of the stent;
wherein each radial axial row is arranged so as to be offset along the longitudinal axis of the stent from each adjacent radial axial row;
wherein each radial support cell is formed from four of the plurality of interconnected struts of the scaffold which are arranged so as to be symmetrical along both the longitudinal axis and the circumferential axis of the stent; and
wherein each flexible support cell is formed from four of the plurality of interconnected struts of the scaffold which are arranged such that two adjacent flexible axial rows have mirror symmetry along the longitudinal axis of the stent.

18. A stent having a substantially tubular body comprising:
a proximal end and an opposing distal end;
a scaffold having a plurality of interconnected struts forming a plurality of radial axial rows of radial support cells and a plurality of flexible axial rows of flexible support cells; and
an at least one radiopaque element formed on the stent;
wherein the plurality of radial axial rows and the plurality of flexible axial rows are disposed in an alternating pattern along a circumferential axis of the stent;
wherein a center axis of each radial axial row and flexible axial row is parallel to a longitudinal axis of the stent;
wherein each radial axial row is arranged so as to be offset along the longitudinal axis of the stent from each adjacent radial axial row; and
wherein each radial support cell is formed from four of the plurality of interconnected struts of the scaffold which are arranged so as to be symmetrical along both the longitudinal axis and the circumferential axis of the stent.
